(19) United States
(12) Patent Application Publication
(10) Pub. No.: US 2019/0022114 A1
(43) Pub. Date: Jan. 24, 2019

(54) METHODS AND COMPOSITIONS FOR REDUCING BODY WEIGHT AND INCREASING GUT MOTILITY

(71) Applicant: SHANGHAI YAO YUAN BIOTECHNOLOGY CO., LTD., Shanghai (CN)

(72) Inventor: Cong XU, Shanghai (CN)

(21) Appl. No.: 16/069,104

(22) PCT Filed: Jan. 11, 2017
(86) PCT No.: PCT/CN2017/070846

§ 371 Date: Jul. 10, 2018

(30) Foreign Application Priority Data
Jan. 11, 2016 (CN) PCT/CN2016/070568

(51) Int. Cl. A61K 31/7004; A61K 9/00; A23L 33/00; A23L 33/125; A61P 3/04; A61P 1/14

(52) U.S. Cl. CPC A61K 31/7004; A61K 9/0095; A23L 33/30; A23L 33/125; A23V 2250/626; A61P 3/04; A61P 1/14; A23V 2002/00; A23L 33/40

(57) ABSTRACT

The present invention relates methods and compositions for reducing body weight and increasing gut motility using D-ribose or an analog or derivative thereof.

### TECHNICAL FIELD

The present invention relates generally to methods and compositions for reducing body weight and increasing gut motility, more particularly, to compositions comprising D-ribose for reducing body weight and increasing gut motility and method of use thereof.

### BACKGROUND OF INVENTION

In the recent decades, overweight and/or obese populations have been steadily rising worldwide, and particularly in the U.S. (Chaudhri, et al., 2005, Drug Discovery Today: Disease Mechanisms 2:289-294; Mokdad, et al., 2003, JAMA 289:76-79; Nguyen and El-Serag, 2 10, Gastroenterol Clin North Am 39:1-7; Wang and Beydoun, 2007, Epidemiol Rev. 29:6-28). Resulting from this is an alarming increase in diabetes, as well as other related health risks that have a significant impact on morbidity and quality of life. Not surprisingly, these consequential health risks incur substantial health and social costs (Kopelman, 2000, Nature 404:635-643; Must, et al., 1999, JAMA 282:1523-1529; Wang, et al., 2008, Obesity 16:2323-2330).

Obesity in China has also become a widespread disease. The etiology of obesity is multifaceted, ranging from genetic factors to environmental influences, such as the adoption of more sedentary lifestyles and the readily available sources of high-calorie food found in modern societies (Bleich, et al., 2008, Annu Rev Public Health 29:273-295; ROssner, 2002, Int J Obes Relat Metab Disord 26(Suppl 4):52-4). The exact mechanisms causing obesity, however, are still not clearly understood.

Currently there are 5 FDA approved anti-obesity drugs, including Xenical, a pancreatic lipase inhibitor, Qsymia, Belviq, and Contrave, agents suppressing appetite via effects on the central nervous system, and Saxenda, an agent acting on glucose metabolism. All these drugs lack strong efficacy (only 3-9% weight loss over 52 weeks) and cause serious side effects, including acute kidney injury, liver damage, headache, etc. Dropout rates for these drugs are up to 50%, mostly resulting from intolerable side-effects.

Worldwide demand for anti-obesity substances has led to research and study of drugs and foods that counteract the progressive body weight accumulation.

D-ribose exists in the following chemical structures:

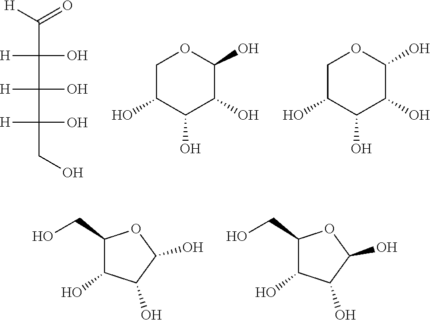 [SMILES: O=CC(O)C(O)C(O)CO ; O[C@@H]1[C@H](O)[C@H](O)CO[C@H]1O ; O[C@@H]1[C@H](O)[C@H](O)CO[C@@H]1O ; OC[C@H]1O[C@H](O)[C@H](O)[C@@H]1O ; OC[C@H]1O[C@@H](O)[C@H](O)[C@@H]1O]

It is a key component of riboflavin (i.e., vitamin B2), ribonucleic acid (RNA), and adenosine tri-phosphate (ATP).

A recent study in rats demonstrated that, administration of D-ribose to rats at a concentration of up to 20% of their diet (15.0 and 15.7 g/kg body weight/day for males and females, respectively) for 13 weeks resulted in a nutritional but not toxicological effect which is likely reversible upon cessation of test diet, and a concentration of 5% D-ribose in the diet (3.6 and 4.4 g/kg body weight/day for males and females, respectively) is deemed as the absolute no observed adverse effect level (NOAEL) for this substance (James C. Griffiths, et. al, Sub-chronic (13-week) oral toxicity study with D-ribose in Wistar rats, Food and Chemical Toxicology 45 (2007) 144-152).

U.S. Pat. No. 6,525,027B2 disclosed the use of ribose supplement in conjunction with weight-training exercise in order to achieve the desired results of increasing muscle mass and decreasing body fat in healthy people more rapidly than with exercise alone.

However, there is no teaching in the prior art that, D-ribose, when administered at high concentration, can reduce body weight in a subject, especially in an overweight and/or obese subject.

### SUMMARY OF THE INVENTION

The present invention provides a method of regulating, in particular, reducing body weight in a subject, comprising administering to the subject a composition comprising an effective amount of D-ribose or an analog or derivative thereof.

The present invention also provides a pharmaceutical and/or food composition for regulating, in particular, reducing body weight in a subject, comprising an effective amount of D-ribose or an analog or derivative thereof as well as a pharmaceutically acceptable carrier or a food additive.

The present invention also relates to the use of D-ribose or an analog or derivative thereof in the manufacture of a pharmaceutical and/or food composition for regulating, in particular, reducing body weight in a subject.

The present invention also relates to D-ribose or an analog or derivative thereof for use in regulating, in particular, reducing body weight in a subject.

The present invention also relates to a pharmaceutical and/or food composition comprising an effective amount of D-ribose or an analog or derivative thereof for use in regulating, in particular, reducing body weight in a subject.

The present invention provides a method of treating or preventing metabolic syndrome, metabolic diseases or disorders, such as diabetes, obesity or overweight in a subject, comprising administering to the subject a composition comprising an effective amount of D-ribose or an analog or derivative thereof.

The present invention also provides a pharmaceutical and/or food composition for treating or preventing metabolic syndrome, metabolic diseases or disorders, such as diabetes, obesity or overweight in a subject, comprising an effective amount of D-ribose or an analog or derivative thereof as well as a pharmaceutically acceptable carrier or a food additive.

The present invention also relates to the use of D-ribose or an analog or derivative thereof in the manufacture of a pharmaceutical and/or food composition for treating or preventing metabolic syndrome, metabolic diseases or disorders, such as diabetes, obesity or overweight in a subject.

The present invention also relates to D-ribose or an analog or derivative thereof for use in treating or preventing metabolic syndrome, metabolic diseases or disorders, such as diabetes, obesity or overweight in a subject.

The present invention also relates to a pharmaceutical and/or food composition comprising an effective amount of D-ribose or an analog or derivative thereof for use in treating or preventing metabolic syndrome, metabolic diseases or disorders, such as diabetes, obesity or overweight in a subject.

The present invention also provides a method of increasing gut motility, in particular, for treating or preventing constipation in a subject, comprising administering to the subject a composition comprising an effective amount of D-ribose or an analog or derivative thereof.

The present invention also provides a pharmaceutical and/or food composition for increasing gut motility, in particular, for treating or preventing constipation in a subject, comprising an effective amount of D-ribose or an analog or derivative thereof as well as a pharmaceutically acceptable carrier or a food additive.

The present invention also relates to the use of D-ribose or an analog or derivative thereof in the manufacture of a pharmaceutical and/or food composition for increasing gut motility, in particular, for treating or preventing constipation in a subject.

The present invention also relates to D-ribose or an analog or derivative thereof for use in increasing gut motility, in particular, for treating or preventing constipation in a subject.

The present invention also relates to a pharmaceutical and/or food composition comprising an effective amount of D-ribose or an analog or derivative thereof for use in increasing gut motility, in particular, for treating or preventing constipation in a subject.

### BRIEF DESCRIPTION OF THE DRAWINGS

FIG. 1, comprising FIGS. 1A and 1B, shows the weight loss effect of D-ribose treatment. FIG. 1A shows the weight loss effect of D-ribose administered at 75 mg/ml concentration in water to LEPR mutants, the D-ribose dosage was 30.03 gram/kg/day.

FIG. 1B shows the weight loss effect of D-ribose administered at 50 mg/ml concentration in water to LEPR mutants, the D-ribose dosage was 20.59 gram/kg/day.

FIG. 2, comprising FIGS. 2A and 2B, shows the D-ribose treatment results in percentage terms for the experiment described in FIG. 1. FIG. 2A shows the treatment results of D-ribose administered at 75 mg/ml concentration in water to LEPR mutants, the D-ribose dosage was 30.03 gram/kg/day. FIG. 2B shows the treatment results of D-ribose administered at 50 mg/ml concentration in water to LEPR mutants, the D-ribose dosage was 20.59 gram/kg/day.

FIG. 3 shows the effect of D-ribose treatment in leading to fat loss.

FIG. 4 shows effect of D-ribose in increasing gut motility. C57BL/6 mice were treated with 0.5 ml 75 mg/ml ribose or xylose. The gut transition time was calculated by counting the time from oral gavage of carmine solution to first red fecal pellet expel. C57BL/6 mice treated with 0.5 ml 75 mg/ml ribose shows shortened gut transition time as compared with control C57BL/6 mice treated with 0.5 ml 75 mg/ml xylose (p value=0.004).

### DETAILED DESCRIPTION

The present invention is based partially on the discovery that, when administered at a relative high strength, for example, in the form of water solution at a concentration of, e.g., about 50 mg/ml or higher, preferably, about 75 mg/ml or higher, D-ribose induces a dosage dependent weight loss and fat loss in obese individuals and increases gut motility.

Therefore, in one aspect, the present invention provides a method of regulating, in particular, reducing body weight in a subject, the method comprises administering to the subject a composition comprising an effective amount of D-ribose or an analog or derivative thereof.

In another aspect, the present invention provides a method of treating or preventing metabolic syndrome, metabolic diseases or disorders, such as diabetes, obesity or overweight in a subject, comprising administering to the subject of a composition comprising an effective amount of D-ribose.

In another aspect, the present invention provides a method of increasing gut motility, in particular, for treating or preventing constipation in a subject, comprising administering to the subject of a composition comprising an effective amount of D-ribose.

In another aspect, the present invention provides a pharmaceutical and/or food composition for regulating, in particular, reducing body weight in a subject, comprising an effective amount of D-ribose or an analog or derivative thereof as well as a pharmaceutically acceptable carrier or a food additive.

In another aspect, the present invention provides a pharmaceutical and/or food composition for treating or preventing metabolic syndrome, metabolic diseases or disorders, such as diabetes, obesity or overweight in a subject, comprising an effective amount of D-ribose or an analog or derivative thereof as well as a pharmaceutically acceptable carrier or a food additive.

In another aspect, the present invention provides a pharmaceutical and/or food composition for increasing gut motility, in particular, for treating or preventing constipation in a subject, comprising an effective amount of D-ribose or an analog or derivative thereof as well as a pharmaceutically acceptable carrier or a food additive.

The present invention also relates to the use of D-ribose or an analog or derivative thereof in the manufacture of a pharmaceutical and/or food composition for regulating, in particular, reducing body weight in a subject.

The present invention also relates to the use of D-ribose or an analog or derivative thereof in the manufacture of a pharmaceutical and/or food composition for treating or preventing metabolic syndrome, metabolic diseases or disorders, such as diabetes, obesity or overweight in a subject.

The present invention also relates to the use of D-ribose or an analog or derivative thereof in the manufacture of a pharmaceutical and/or food composition for increasing gut motility, in particular, for treating or preventing constipation in a subject.

In one embodiment, the subject is a mammal, and more preferably, a human

In one embodiment, the subject is, for example, but not limited to, an overweight and/or obese subject, a diabetic subject, and the like.

The composition of the present invention may be selected from the group consisting of a pharmaceutical compositions, food compositions, pharmaceutical preparations, nutritional compositions, nutraceuticals, powdered nutritional products to be reconstituted in water or milk before consumption, food supplements, drinks, dietary supplements, functional foods, full meals, desserts, biscuits, energy bars, meal replacements, concentrates or solutions or beverages in ready to drink form, pet food, and the like.

The composition of the present invention may be in solid, semi-solid or liquid form. The composition of the present invention may optionally comprise one or more other therapeutic agents, or one or more nutrients or foods nutritional supplements.

The pharmaceutical and/or food composition of the present application may be a liquid composition, e.g., a water solution, a beverage composition comprising a relative high concentration of D-ribose or an analog or derivative thereof, for example, at a concentration of about 50 mg/ml or higher, about 60 mg/ml or higher and more preferably, about 75 mg/ml or higher.

The liquid compositions set forth herein include concentrates, which may be diluted prior to being consumed, as well as solutions or beverages in a ready-to-drink form.

The pharmaceutical and/or food composition of the present application can also be a solid product that can be easily reconstituted into a water solution comprising a relative high concentration of D-ribose or an analog or derivative thereof, for example, at a concentration of about 50 mg/ml or higher, about 60 mg/ml or higher, and more preferably, about 75 mg/ml or higher. The solid pharmaceutical and/or food composition may also comprise an adjuvant that facilitates the dissolution of D-ribose or an analog or derivative thereof in water.

The solid compositions set forth herein include pharmaceutical preparations, nutritional compositions, nutraceuticals, powdered nutritional products to be reconstituted in water or milk before consumption, food supplements, dietary supplements, functional foods, full meals, desserts, biscuits, energy bars, meal replacements, etc.

The composition of the present invention can be used to reduce body weight without having to reduce food intake.

Hence, weight loss can be achieved while maintaining a food intake that satisfies the body's needs. Consequently, the risk to provide insufficient supply of nutrients while reducing food intake is avoided.

Reducing body weight helps to reduce the risk for developing metabolic syndrome or metabolic diseases or disorders, for example, diabetes type 2.

Any metabolic diseases or disorders may be treated or prevented according to the present invention. For example, the metabolic diseases or disorders may be selected from the group consisting of diabetes, hypertension, cardiovascular diseases, and combinations thereof.

Definitions:

As used herein, each of the following terms has the meaning associated with it in this section.

The articles “a” and “an” are used herein to refer to one or to more than one (i.e. to at least one) of the grammatical object of the article. By way of example, “an adjuvant” means one adjuvant or more than one adjuvants.

The term “about” will be understood by persons of ordinary skill in the art and will vary to some extent on the context in which it is used. Preferably, the term “about” is intended to modify a numerical value above and below the stated value by a variance of ≤20%, more preferably ≤10%.

As used herein, the term “treatment” or “treating” is defined as the application or administration of a therapeutic agent, i.e., a compound, such as D-ribose an analog or derivative thereof, useful within the invention (alone or in combination with another agent, for example, pharmaceutically acceptable carrier or adjuvant), to a subject, or application or administration of a therapeutic agent to an isolated tissue or cell either engineered or from a subject (e.g., for diagnosis or ex vivo applications), with the purpose to cure, heal, alleviate, relieve, alter, remedy, ameliorate, improve or affect the condition being treated, for example, the metabolic syndrome disease or disorder, or reduce the body weight of a subject.

As used herein, the term “patient” or “subject” refers to a human or a non-human animal. Non-human animals include, for example, livestock and pets, such as ovine, bovine, porcine, canine, feline and murine mammals. Preferably, the patient or subject is a mammal, and more preferably, a human

As used herein, the terms “effective amount,” “pharmaceutically effective amount” and “therapeutically effective amount” refer to a non-toxic but sufficient amount of an agent to provide the desired biological result. That result can be reduction and/or alleviation of the signs, symptoms, or causes of a disease, or any other desired alteration of a biological system, for example, the reduction of the body weight of an overweight or obese subject. An appropriate therapeutic amount in any individual case may be determined by one of ordinary skill in the art using routine experimentation. Typically, the effective amount of D-ribose or an analog or derivative thereof for a adult of about 75 kg is about 20-500 grams/day, for example about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 grams/day.

As used herein, the term “composition” or “pharmaceutical/food composition” refers to a mixture of at least one compound useful within the invention with a pharmaceutically acceptable carrier or a food additive, when appropriate. The pharmaceutical composition facilitates administration of the compound to a patient. Multiple techniques of administering a compound exist in the art including, but not limited to, intravenous, intraperitoneally, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “a food additive” includes one or more of the following: acidulants, additional thickeners, buffers or agents for pH adjustment, chelating agents, colorants, emulsifiers, excipients, flavor agents, minerals, osmotic agents, preservatives, stabilizers, sugar, sweeteners, texturizers, vitamins, etc.

D-ribose exists as the following chemical structures:

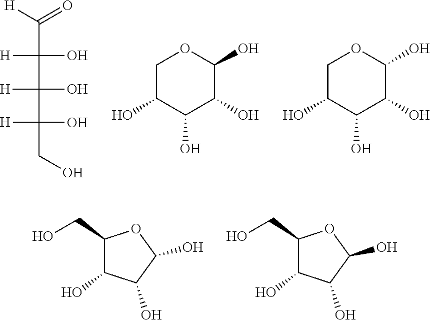 [SMILES: O=CC(O)C(O)C(O)CO ; O[C@@H]1[C@H](O)[C@H](O)CO[C@H]1O ; O[C@@H]1[C@H](O)[C@H](O)CO[C@@H]1O ; OC[C@H]1O[C@H](O)[C@H](O)[C@@H]1O ; OC[C@H]1O[C@@H](O)[C@H](O)[C@@H]1O]

It may be provided by any means known to those of skill in the art. They may be synthesized chemically or may be provided from natural sources, e.g., in purified form or in the form of an extract.

As described herein, “D-ribose or an analog or derivative thereof” means D-ribose per se and analog or derivative of the D-ribose having similar body weight reducing activity. D-ribose per se exists as the open-chain form and also the two cyclic forms. the pro-drugs of D-ribose are compounds having acyclic and cyclic structures wherein one or more hydroxy groups being etherized with lower alkyl or alkylene, e.g., C1-C6 alkyl or C1-C6 alkylene, with benzyl or aromatic rings, or heterocycles. The derivatives cover the molecules of removing any hydroxy group on the cyclic ring and the acyclic ring and the left hydroxy groups being etherized with lower alkyl or alkylene, e.g., C1-C6 alkyl or C1-C6 alkylene, with benzyl or aromatic rings, or heterocycles. Both the pyran and furan type isomers and their hydroxy decrease analogs (cyclic ring structures) are esterified with organic or inorganic acids or amino acids, e.g., C1-C6 alkane carboxylic acids such as formic acid, acetic acid, sulphuric acid, phosphate acid and phosphate acid analogs, and essential amino acids well known in the art, for example, ribose-5-phosphate. The sodium salt of both pyran and furan isomers are included as well.

The molecule with the substitution of the any hydroxy group with F, Cl or NH₂, and the substitution of the O in the pyran and furan rings with S, NH and N analogs, are also dedicated as the D-ribose derivatives herein.

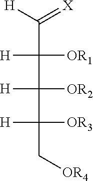

R1, R2, R3, R4 are H, alkyl, alkylene, benzyl, aromatic rings or heterocycles, phosphate, sulfuric, acetic acid ester, other organic and inorganic acid;

X═O, S, NH or N-analogs

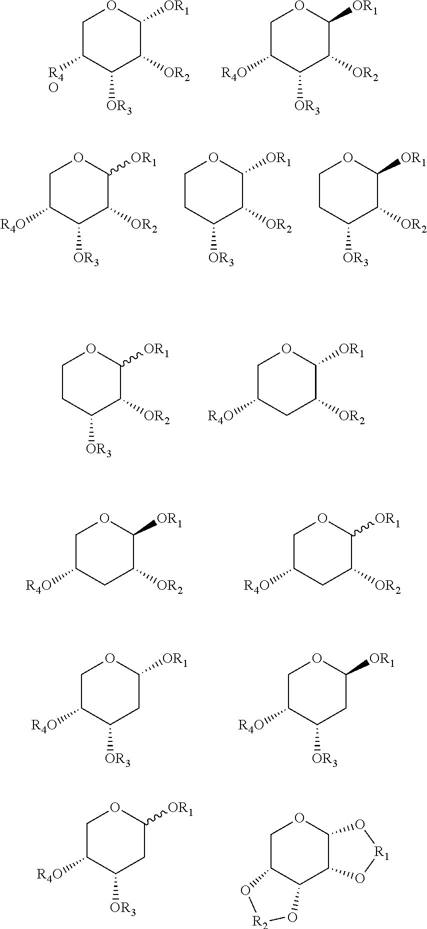

R1, R2, R3, R4 are H, alkyl, alkylene, benzyl, aromatic rings or heterocycles, phosphate, sulfuric, acetic acid ester, other organic and inorganic acid.

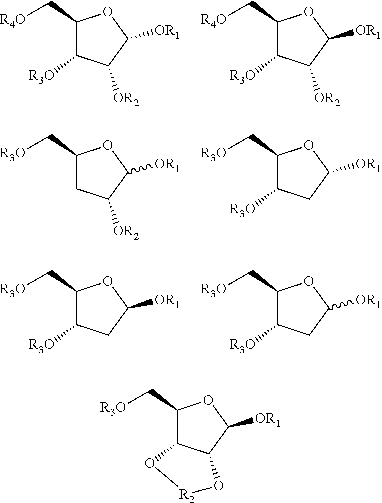

R1, R2, R3, R4 are H, alkyl, alkylene, benzyl, aromatic rings or heterocycles, phosphate, sulfuric, acetic acid ester, other organic and inorganic acid.

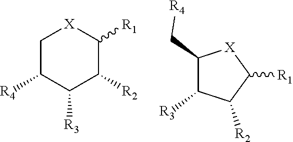

R1, R2, R3 and R4 are H, hydroxy, amino, or F atoms

X=O, C, S, or NH.

“Overweight” is defined, for example, for an adult human as having a BMI between 25 and 30.

“Body mass index” or “BMI” means the ratio of weight in kg divided by the height in metres, squared.

“Obesity” is a condition in which the natural energy reserve, stored in the fatty tissue of animals, in particular humans and other mammals, is increased to a point where it is associated with certain health conditions or increased mortality. “Obesity” is defined, for example, for an adult human as having a BMI greater than 30.

Compositions

The invention includes a pharmaceutical or food composition comprising D-ribose, or an analog or derivative thereof. In one aspect of the invention, the composition further comprises a pharmaceutically acceptable carrier.

In one embodiment, the compositions of the invention are formulated using one or more pharmaceutically acceptable excipients or carriers. In one embodiment, the pharmaceutical compositions of the invention comprise a therapeutically effective amount of D-ribose or an analog or derivative thereof and one or more pharmaceutically acceptable carrier.

The compositions can be used to administer D-ribose or an analog or derivative thereof to a cell, a tissue, an organ or an animal. The compositions are useful to treat or prevent a disease, disorder or condition such that weight loss is beneficial. That is, where a disease, disorder or condition in an animal is mediated by or associated with weight change, a composition of the present invention can be used to regulate such activity.

In a particular embodiment, the present composition can be used to reduce the body weight in an overweight or obese subject.

In another particular embodiment, the present composition can be used to treat or prevent metabolic syndrome, metabolic diseases or disorders, such as diabetes, obesity or overweight in a subject.

The pharmaceutical and/or food composition of the present application can be a liquid composition comprising a relative high concentration of D-ribose or an analog or derivative thereof, for example, at a concentration of about 50 mg/ml or higher, and more preferably, about 75 mg/ml or higher. The liquid compositions set forth herein include concentrates as well as solutions or beverages in ready to drink form.

The pharmaceutical and/or food composition of the present application can also be a solid composition that can be easily reconstituted into a liquid composition by mixing with, e.g., water or other liquids, prior to being consumed. The solid compositions set forth herein include pharmaceutical preparations, nutritional compositions, nutraceuticals, powdered nutritional products to be reconstituted in water or milk before consumption, food supplements, dietary supplements, functional foods, full meals, desserts, biscuits, energy bars, meal replacements etc.

The compounds, for example, the D-ribose derivatives described herein, may form salts with acids, and such salts are included in the present invention. In one embodiment, the salts are pharmaceutically acceptable salts. The term “salts” embraces addition salts of free acids that are useful within the methods of the invention. The term “pharmaceutically acceptable salt” refers to salts that possess toxicity profiles within a range that affords utility in pharmaceutical applications.

In addition, the compositions of the invention may be formulated as therapeutic compositions for treatment of disease states, or may be formulated as a neutraceutical preparation, suitable for inclusion in beverages, foods, and the like.

Administration/Dosage/Formulations

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other body weight reducing agents, e.g., a pancreatic lipase inhibitor, agents suppressing appetite via effects on the CNS, such as Xenica, Qsymia, Belviq, Contrave and Saxenda. The formulation of the present invention may also be used in conjunction with other means of reducing body weight, for example, physical exercise.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

In one embodiment, the present composition is administered orally in the form of a liquid composition.

In another embodiment, the composition of the present application is administered orally in the form of a solid composition. The solid compositions set forth herein include pharmaceutical preparations, nutritional compositions, nutraceuticals, powdered nutritional products to be reconstituted in water or milk before consumption, food supplements, dietary supplements, functional foods, full meals, desserts, biscuits, energy bars, meal replacements, etc. The content of D-ribose or an analog or derivative thereof in said solid composition can be in the range of from 1 to 100%, e.g., 10 to 20% by weight of the composition.

The regimen of administration may affect what constitutes an effective amount. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Actual dosage levels of D-ribose or an analog or derivative thereof in the pharmaceutical compositions of the present invention may be varied so as to obtain an amount of the active ingredient that is effective to achieve the desired therapeutic response for a particular subject, composition, and mode of administration, without being toxic to the subject.

In one embodiment, D-ribose or an analog or derivative thereof is administered in the form of a liquid or solid composition in an amount of about 20-500 grams/day, for example about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 grams/day.

Administration of the compositions of the present invention to a subject, preferably a mammal, more preferably a human, may be carried out using known procedures, at dosages and for periods of time effective to reduce the body weight of the subject, especially an overweight or obese subject. An effective amount of the therapeutic compound necessary to achieve the desired effect may vary according to factors such as the age, sex, and weight of the subject. Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of the therapeutic compound without undue experimentation.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values, in whole or partial increments, that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

Preparation of the Solution of D-Ribose

D-ribose is purchased from Jiangsu Cheng Zhi Sheng Wu and used directly without any further purification.

Water solutions of D-ribose at concentrations indicated (75 mg/ml and 50 mg/ml) were prepared by dissolving suitable amount of D-ribose in distilled water.

### Example 2

Weight Loss Effect of D-Ribose in Mouse Obesity Models

Materials and Methods

Classic obesity model LEPR (Leptin Receptor) mutants (Age 42 day, male) and wild-type FVB strain mice were used in the testing.

D-ribose was feed to mice in D-ribose water solution at concentrations indicated (75 mg/ml and 50 mg/ml). Control animals were feed with water. All animals were fed on normal diet. There was no restriction on the amount of liquid or food consumption.

The liquid and food consumption were measured at 4 day intervals. The actual D-ribose consumption for each animal was calculated based on its liquid intake volume.

Mouse body fat was measured using MiniSpec NMR Analyzer.

P-values were calculated using t-test (unpaired, 2 tails) for data points after 48 days of treatment.

Results

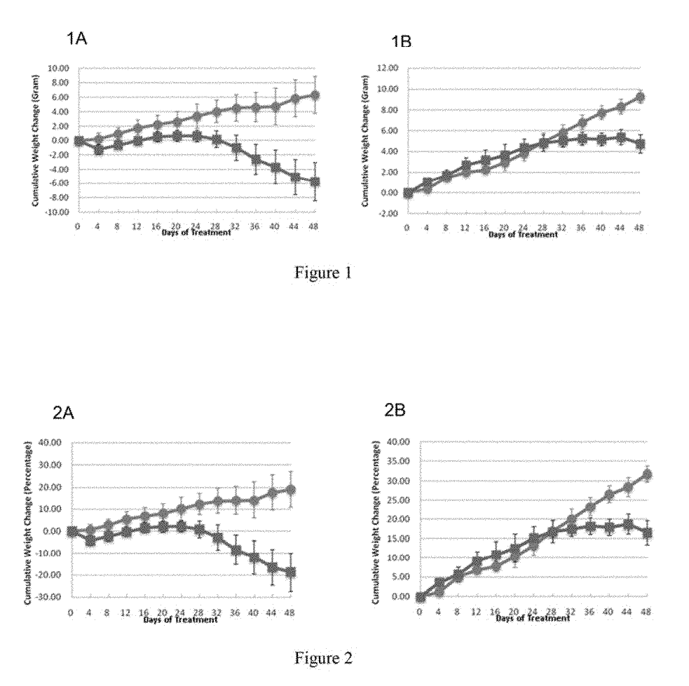

Test results were shown in FIG. 1 and FIG. 2, wherein Square (▪) represents the treatment group, and Circle (●) represents control group. Classic obesity model LEPR (Leptin Receptor) mutants (Age 42 day, male) were used in the testing. The control group was fed with water. Animals were fed on normal diet.

D-Ribose Treatment at 75 mg/ml Concentration Led to Statistically Significant Weight Loss and Fat Loss in Obese Mice.

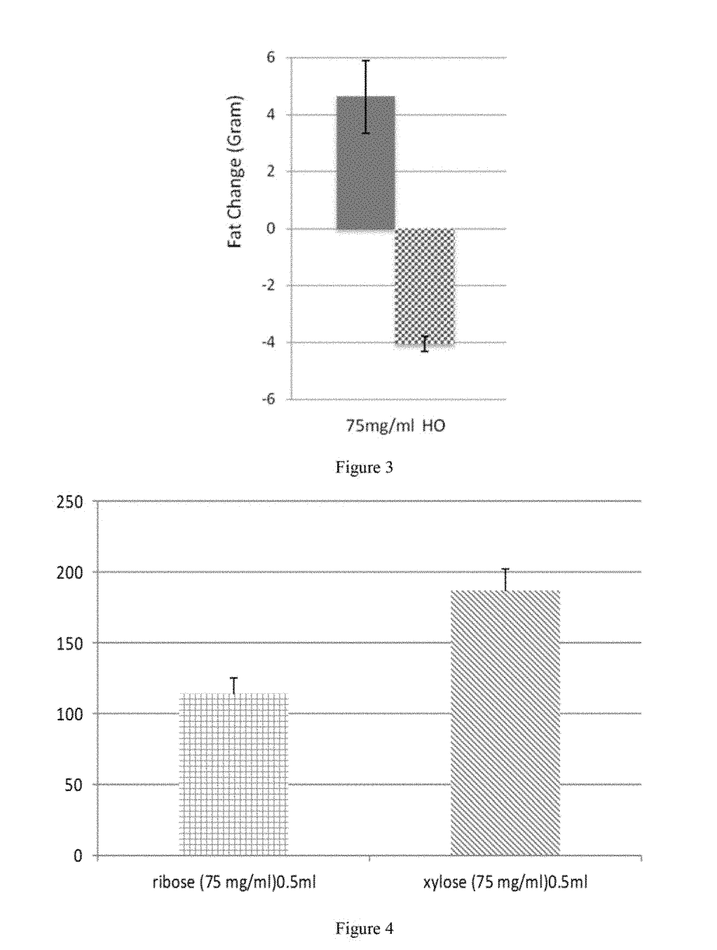

When D-ribose was fed to obese mice (6 week old LEPR mutant male mice) as a water solution at 75 mg/ml concentration, it led to statistically significant weight loss (p-value 0.01, FIG. 1A and FIG. 2A) and fat loss (p-value 0.00015, FIG. 3) as compared with the control animals after 48 days of treatment. The actual D-ribose dosage was 30.03 gram/kg/day. The weight loss was significant both in absolute weight (FIG. 1A) and percentage terms (FIG. 2A).

D-Ribose Treatment at 50 mg/ml Concentration Led to Statistically Significant Decrease of Weight Gain in Obese Mice.

When D-ribose was fed to obese mice (6 week old LEPR mutant male mice) as a water solution at 50 mg/ml concentration, it led to statistically significantly decreased weight gain (p-value 0.001, FIG. 1B) as compared with the control animals after 48 days of treatment. The actual D-ribose dosage was 20.59 gram/kg/day. Body fat measurement found that D-ribose also reduced fat gain but not statistically significant (data not shown).

Fat Loss Effect of D-Ribose Treatment

FIG. 3 shows the fat loss effect of D-ribose treatment. D-ribose was administered to LEPR (Leptin Receptor) mutants (6 week old, male) at 75 mg/ml concentration in water solution. Solid: Control. Checkered: Treatment. HO: homozygous LEPR mutants. The D-ribose dosage was 30.03 gram/kg/day. The body composition of each animal was measured before and after the 48-day treatment using NMR machine. Body fat increased in the control group while statistically significantly decreased in the treated group. (P-value: 0.00015).

The above test data indicated that when fed at high concentration (75 mg/ml water solution), D-ribose led to statistically significant weight loss and fat loss in obese individuals. When fed at medium concentration (50 mg/ml water solution), the effect of D-ribose was less significant but still led to a decrease of weight gain. The dosage dependence of the effect suggests that the weight loss observed was caused by D-ribose.

Gut Motility Increase Effect of D-Ribose Treatment

D-ribose was administered to C57BL/6 mice of 8 weeks old. Before administering, mice were fasted for 18 hours with free access to water and housed on a mesh plate without bedding. The mice were orally fed with 0.5 ml carmine solution (6% carmine in 0.5% methylcellulose) containing either 75 mg/ml D-ribose or xylose. After oral force gavage, mice were returned to individually housed mesh bottom cages, which were placed on a white sheet. Whole gut transit time is the time taken from carmine meal gavage to the appearance of the first red fecal pellets.

FIG. 4 shows effect of D-ribose in increasing gut motility. C57BL/6 mice treated with 0.5 ml 75 mg/ml ribose shows shortened gut transition time as compared with control C57BL/6 mice treated with 0.5 ml 75 mg/ml xylose (p value=0.004).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of reducing body weight in a subject comprising administering to subject in need thereof a composition comprising an effective amount of D-ribose or an analog or derivative thereof

2. A method of treating constipation metabolic syndrome, metabolic diseases or disorders comprising administering to a subject in need thereof a composition comprising an effective amount of D-ribose or an analog or derivative thereof.

3. (canceled)

4. The method according to claim 2, wherein the subject is a mammal.

5. The method according to claim 2, wherein the D-ribose or an analog or derivative thereof is administered in an amount of about 20-500 grams/day.

6. The method according to claim 2, wherein the subject is an overweight and/or obese subject, or a diabetic subject.

7. The method according to claim 2, wherein the composition is administered as a liquid composition comprising a relative high concentration of D-ribose or an analog or derivative thereof at a concentration of about 50 mg/ml or higher.

8. The method according to claim 2, wherein the composition is administered in the form of a solid composition.

9. A pharmaceutical and/or food composition comprising an effective amount of D-ribose or an analog or derivative thereof as well as a pharmaceutically acceptable carrier or a food additive.

10. The pharmaceutical and/or food composition according to claim 9, wherein the composition may be in the form of solid, semi-solid or liquid.

11. The pharmaceutical and/or food composition according to claim 9, wherein the composition is a liquid composition comprising D-ribose or an analog or derivative thereof at a concentration of about 50 mg/ml or higher.

12. The pharmaceutical and/or food composition according to claim 9, wherein the composition is a solid composition comprising D-ribose or an analog or derivative thereof in the range of from 1 to 100%.

13. -16. (canceled)

17. The method according to claim 2, wherein the metabolic syndrome, metabolic disease or disorder is diabetes, obesity, or overweight.

18. The method according to claim 2, wherein the subject is a human.

19. The method according to claim 2, wherein the D-ribose is administered in a dose selected from the group consisting of about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 grams/day.

20. The method according to claim 7, wherein the concentration is about 60 mg/ml or higher.

21. The method according to claim 7, wherein the concentration is about 75 mg/ml or higher.

22. (canceled)

23. The pharmaceutical and/or food composition according to claim 10, wherein the composition is in a form selected from the group consisting of pharmaceutical preparations, nutritional compositions, nutraceuticals, powdered nutritional products to be reconstituted in water or milk before consumption, food supplements, drinks, dietary supplements, functional foods, full meals, desserts, biscuits, energy bars, meal replacements, concentrates or solutions or beverages in ready to drink form, and pet food.

24. The pharmaceutical and/or food composition according to claim 11, wherein the D-ribose or an analog or derivative thereof is at a concentration of about 60 mg/ml or higher.

25. The pharmaceutical and/or food composition according to claim 11, wherein the D-ribose or an analog or derivative thereof is at a concentration of about 75 mg/ml or higher.

26. The pharmaceutical and/or food composition according to claim 9, wherein the composition is a solid composition comprising D-ribose or an analog or derivative thereof in the range of from 10 to 20% by weight of the composition.
